(19) United States
(12) Patent Application Publication
(10) Pub. No.: US 2004/0253186 A1
(43) Pub. Date: Dec. 16, 2004

(54) Use of a polysiloxane sunscreen to enhance fragrance retention on hair

(76) Inventors: Philippe Emmanuel Maillan, Eschentzwiller (FR); Horst Westenfelder, Neustadt a.d.W. (DE)

Correspondence Address: Stephen M Haracz, Bryan Cave, 1290 Avenue of the Americas, New York, NY, 10104-3300, US

(21) Appl. No.: 10/493,564

(22) PCT Filed: Oct. 17, 2002
(86) PCT No.: PCT/EP02/11600

(30) Foreign Application Priority Data
Oct. 24, 2001 (EP) 011251873

(51) Int. Cl. A61K 9/00; A61K 7/42

(52) U.S. Cl. 424/47; 424/59

(57) ABSTRACT

The use of a polysiloxane sunscreen as a fragrance fixating agent in hairspray compositions.

The present invention relates to an improvement in fragrance retention on hair treated with hairspray compositions.

Perfumes are a conventional component of hairsprays. In accordance with the present invention it has been found that polysiloxane sunscreens improve the retention on hair of fragrances contained in hairsprays.

Accordingly, the present invention relates to the use of a polysiloxane sunscreen as a fragrance fixating agent in hairspray compositions. The polysiloxane sunscreen may be any conventional polysiloxane sunscreen as disclosed, e.g., in EP 392,882, EP 358,584, EP 538,431, and EP 709,080. Examples of such polysiloxane sunscreens are those of the general formula Ia or Ib

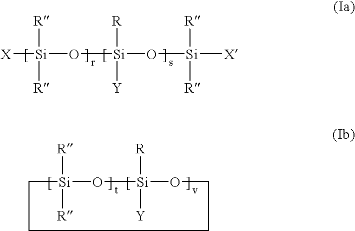

wherein

R and R″ are C₁₋₁₀alkyl or phenyl;

X and X′ are C₁₋₁₀alkyl, phenyl or a group Y;

r is an average number from 0 to 150;

s is an average number from 0 to 30; where, when s=0 at least one of X and X′ is Y;

the sum of r and s is ≧3;

t is an average number from 0 to 10;

v is an average number from 1 to 10;

the sum of v and t is ≧3;

Y is a group of the formula (a) or (b)

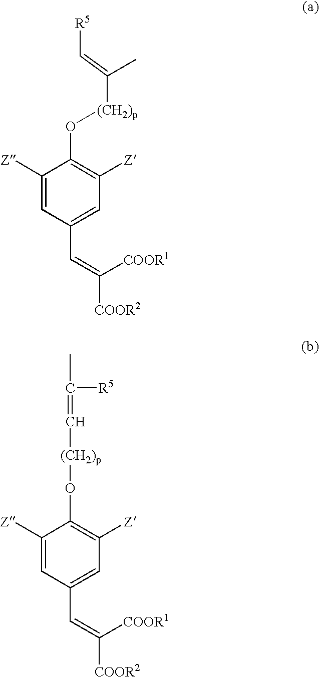

wherein

R¹ and R² are C₁₋₆alkyl;

R⁵ is hydrogen or C₁₋₆alkyl;

Z′ and Z″ are, independently, hydrogen, hydroxy, C₁₋₆alkyl or C₁₋₆alkoxy, and

p is an integer from 1 to 6;

or a group of the formula (c)

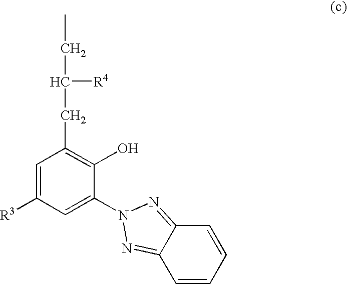

wherein R³, R⁴ is hydrogen or C₁₋₆-alkyl.

Compounds of the formula Ia and Ib wherein Y is a group (a) or (b) and their preparation are disclosed, e.g., in EP 538,431 and EP 679,645. Compounds of the formula Ia or Ib wherein Y is a group (c) and their preparation are disclosed, e.g., in EP 354,145, EP 388,218, EP 392,883 and WO 94/06,404.

Preferred are polysiloxane sunscreens of the formula Ia wherein s is an average number of 3 to 10 and r is an average number of 20 to 80. Most preferred are polysiloxane sunscreens of the formula Ia wherein s is ca. 4 and r is ca. 60, and wherein about 80% of the groups Y are of the alkylidene structure (a) and about 20% of the groups Y are of the alkylene structure (b), such as disclosed in EP 709,080 and referred to therein as “Polysiloxane A”.

Particularly preferred for the purpose of the present invention is the polysiloxane sunscreen of the formula

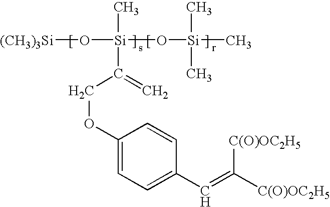

wherein s is ca. 4 and r is ca. 60, and which contains ca. 20% of the alkylene isomer (cf. the general formula (b) above) and which is referred to hereinafter as PARSOL® SLX. The preparation of the polysiloxane sunscreen PARSOL® SLX is disclosed in more detail in EP 709 080 where it is referred to as “Polysiloxane A”.

The amount of the polysiloxane sunscreen in a hairspray for use in accordance with the present invention is about 0.01 to about 10 wt.-%, preferably about 0.1 to about 5 wt.-%.

The perfume can be any perfume or mixture thereof from natural or synthetic origin including essential oils especially those which conventionally find use in hairspray compositions and maybe present in the hairspray composition in an amount of e.g., 0.001 to 50 wt.-%, particularly about 0.01 to about 10 wt.-%, and preferably about 0.1 to about 5 wt.-%.

The hairspray composition can be a non-aerosol spray lotion which is sprayed mechanically or an aerosol which is sprayed with a propellant. Further, the hairspray composition may contain, besides the perfume and the polysiloxane sunscreen fragrance fixation agent additional components conventionally used in hairspray compositions such as vitamins, e.g. biotin, vitamin E or phytantriol; propellants, e.g. DME, F 152, propane, butane or pentane; conventional cosmetic solvents such as ethanol and water; styling polymers; silicones for hair care such as dimethicone, phenyltrimethicone or silicone emulsions; neutralizing agents, e.g. AMP; preservatives; and further UV-A and UV-B absorbing agents, e.g. dibenzoylmethane, cinnamate, camphor or phenylbenzimidazol derivatives to name a few as specified in EP 979 645.

The following Examples illustrate the invention further.

### EXAMPLE 1

### Propellant Spray

| | | % |
|---|---|---|
| A) | Luviflex Silk | 4.80 |
| | Ethanol 96% | 50.63 |
| | Luviskol Plus | 1.50 |
| | AMP | 0.57 |
| | Perfume | 1.00 |
| | PARSOL ® SLX | 1.00 |
| | Panthenol | 0.50 |
| B) | Propane/Butane | 40.00 |
| | | 100.00 |

A solution of phase A) is filled in an aerosol can with phase B) as a propellant.

### EXAMPLE 2

### Propellant Spray

| | | wt.-% |
|---|---|---|
| A) | Amphomer | 5.00 |
| | AMP | 0.82 |
| | Ethanol | 66.18 |
| | PARSOL ® SLX | 2.00 |
| | Perfume | 1.00 |
| B) | Propane/Butane | 25.00 |
| | | 100.00 |

The solution of phase A) is filled in an aerosol can with phase B) as a propellant.

### EXAMPLE 3

### Propellant Spray

| | | wt-% |
|---|---|---|
| A) | Ethanol 96% | 50.50 |
| | AMP | 0.50 |
| | Perfume | 0.50 |
| | Panthenol | 0.50 |
| | Cyclomethicone | 1.00 |
| | PARSOL ® SLX | 1.00 |
| B) | Luviset CA 66 | 6.00 |
| C) | DME | 40.00 |
| | | 100.00 |

Phase B) is added to the solution of phase A) and the mixture is filled in an aerosol can with phase C) as a propellant.

### EXAMPLE 4

### Pump Spray

| | | wt.-% |
|---|---|---|
| A) | Ethanol | 88.20 |
| | Perfume | 2.00 |
| | Amphomer | 2.00 |
| B) | AMP 95% | 0.35 |
| | Vitamine PP | 0.20 |
| | Phytantriol | 0.25 |
| | Ethylpanthenol | 1.00 |
| | PARSOL ® SLX | 5.00 |
| | Phenyltrimethicone | 1.00 |
| | | 100.00 |

The solution of phase A) is mixed with phase B) and the mixture is filled in a pump spray can.

### EXAMPLE 5

### Sensory Panel Evaluation of Fragrance Fixation by PARSOL®SLX

Commercially available, bleached blond hair, flat swatches, 10 cm long were used. The tresses were pre-washed with a commercial shampoo prior to use.

Each hair tress was immersed in 20.0 grams of the spray in a 50 ml glass cylinder. The cylinder was shaken for 1 minute. The tresses were removed, suspended for 2 minutes, combed 10 times from root to end in a downward motion to remove the excess solution, and allowed to dry in ambient conditions for 15 minutes. The hair tresses were then submitted for fragrance evaluation.

The following spray formulas were tested:

| | Ethanol | Fragrance | Fragrance | PARSOL ® |
|---|---|---|---|---|
| Formula | [%] | A [%] | B [%] | SLX [%] |
| 1 | 99.5 | 0.5 | — | — |
| 2 | 98.5 | 0.5 | — | 1.0 |
| 3 | 99.5 | — | 0.5 | — |
| 4 | 98.5 | — | 0.5 | 1.0 |

Fragrance A: “Lemarome” (contains only one fragrance)
Fragrance B: “Canangan” (a complex mixture of fragrances)
Both Fragrances were supplied from Givaudan Roure

A panel consisting of 10 persons were asked to compare 2 tresses each (Placebo and Test) in regard to fragrance intensity. The perceived intensity was plotted on a 10 cm horizontal line by positioning a mark on the line, with the start of the line (0 cm) representing no perceptible odour and the end of the line (10 cm) representing a strong odour. The position of the mark was then read into a numerical value between 0 and 10. The individual ratings and the average of the perceived intensity along with the uncertainty at the P=95% level are shown in tables 1 and 2 below.

TABLE 1
Results obtained with Fragrance A (Lemarome)

| | Formula 1 | Formula 2 | |
|---|---|---|---|
| Test | reported | reported | change |
| person | value | value | [%] |
| 1 | 2.4 | 6.8 | 183 |
| 2 | 4.6 | 0.6 | −87 |
| 3 | 9.3 | 0.7 | −92 |
| 4 | 2.6 | 7.6 | 192 |
| 5 | 1.9 | 3.8 | 100 |
| 6 | 1.5 | 8.0 | 433 |
| 7 | 0.5 | 6.1 | 1120 |
| 8 | 1.4 | 0.9 | −36 |
| 9 | 1.1 | 5.0 | 355 |
| 10 | 1.9 | 5.3 | 179 |
| Average | 2.72 | 4.48 | 235 |
| Standard | 2.56 | 2.86 | — |
| deviation | | | |
| Uncertainty | 1.83 | 2.05 | — |

Uncertainty (P = 95%, n = 10, Student distribution)

As can be seen from Table 1, hair treated with 1% PARSOL®SLX (Formula 2 hair tress) was rated significantly higher in terms of perceived fragrance intensity than the placebo (Formula 1) hair. Further, seven out of ten persons reported that hair treated with PARSOL®SLX had a stronger fragrance. On average, the rating of the fragrance intensity of the PARSOL®SLX treated hair was 2.4 times higher than the rating for placebo.

TABLE 2
Results obtained with Fragrance B (Canangan):

| | Formula 3 | Formula 4 | |
|---|---|---|---|
| Test | reported | reported | change |
| person | value | value | [%] |
| 1 | 6.7 | 9.0 | 34 |
| 2 | 3.4 | 7.0 | 106 |
| 3 | 3.1 | 5.2 | 68 |
| 4 | 2.4 | 5.6 | 133 |
| 5 | 3.5 | 9.2 | 163 |
| 6 | 8.6 | 3.8 | −56 |
| 7 | 2.4 | 9.6 | 300 |
| 8 | 2.6 | 5.7 | 119 |
| 9 | 1.2 | 6.3 | 425 |
| 10 | 5.4 | 7.2 | 33 |
| Average | 3.93 | 6.86 | 133 |
| Standard | 2.28 | 1.92 | |
| deviation | | | |
| Uncertainty | 1.63 | 1.37 | |

Uncertainty (P = 95%, n = 10, Student distribution)

As can be seen from Table 2, hair treated with 1% PARSOL®SLX (Formula 2 hair tress) was rated significantly higher in terms of perceived fragrance intensity than the placebo (Formula 1) hair. Further, nine out of ten persons reported that hair treated with PARSOL®SLX had a stronger fragrance. On average, the rating of the fragrance intensity of the PARSOL®SLX treated hair was 1.3 times higher than the rating for placebo.

1. A method for fixing a fragrance on a hair comprising applying to a hair a composition comprising a polysiloxane sunscreen and a fragrance.

2. A method according to claim 1 wherein the polysiloxane sunscreen is of the formula Ia or Ib 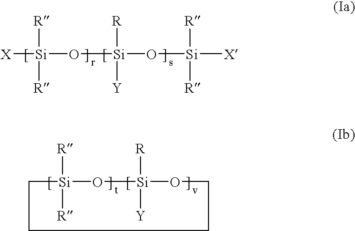
wherein
R and R″ are C₁₋₁₀alkyl or phenyl;
X and X′ are C₁₋₁₀alkyl, phenyl or a group Y;
r is an average number from 0 to 150;
s is an average number from 0 to 30; where, when s=0 at least one of X and X′ is Y;
the sum of r and s is ≧3;
t is an average number from 0 to 10;
v is an average number from 1 to 10;
the sum of v and t is ≧3;
Y is a group of the formula (a) or (b) 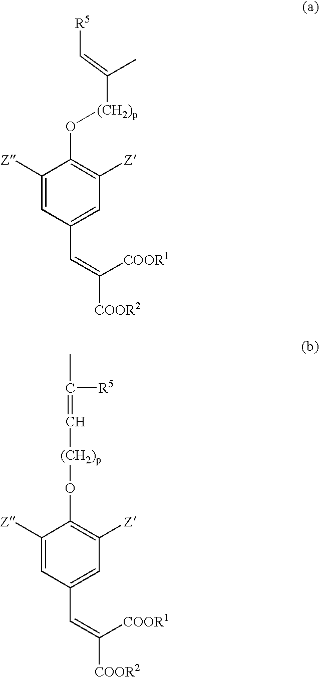
wherein
R¹ and R² are C₁₋₆alkyl;
R⁵ is hydrogen or C₁₋₆alkyl;
Z′ and Z″ are, independently, hydrogen, hydroxy, C₁₋₆alkyl or C₁₋₆alkoxy; and
p is an integer from 1 to 6;
or a group of the formula (c) 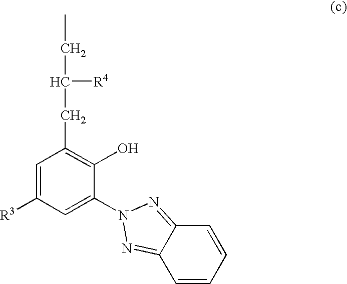
wherein R³, R⁴ is hydrogen or C₁₋₆-alkyl.

3. A method according to claim 1 or 2 wherein the polysiloxane sunscreen is of the formula 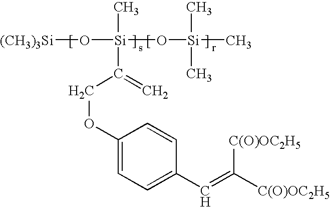
wherein s is about 4 and r is about 60, and which contains about 20% of the alkylene isomer of formula (b).

4. A method for fixing a fragrance on hair comprising applying to a hair a composition comprising a polysiloxane sunscreen and a fragrance, wherein the sunscreen absorbs in a range selected from the group consisting of UV-A, UV-B, broadband and mixtures thereof.

5-6. (Canceled).

7. A method according to claim 1 wherein the amount of the polysiloxane sunscreen in the composition is from about 0.01 to about 10 wt.-% of the total weight of the composition.

8. A method according to claim 2 wherein the amount of the polysiloxane sunscreen in the composition is from about 0.01 to about 10 wt.-% of the total weight of the composition.

9. A method according to claim 3 wherein the amount of the polysiloxane sunscreen in the composition is from about 0.01 to about 10 wt.-% of the total weight of the composition.

10. A method according to claim 4 wherein the amount of the polysiloxane sunscreen in the composition is from about 0.01 to about 10 wt.-% of the total weight of the composition.

11. A method according to claim 1 wherein the applying step comprises spraying the composition onto the hair.

12. A method of making a composition for fixing a fragrance to a hair comprising combining a polysiloxane-containing sunscreen with a fragrance.

13. A method according to claim 12 wherein the sunscreen is of the formula Ia or Ib 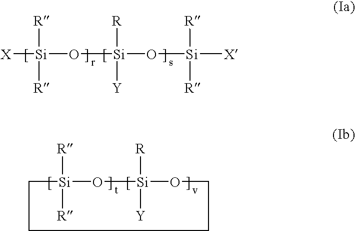
wherein
R and R″ are C₁₋₁₀alkyl or phenyl;
X and X′ are C₁₋₁₀alkyl, phenyl or a group Y;
r is an average number from 0 to 150;
s is an average number from 0 to 30; where, when s=0 at least one of X and X′ is Y;
the sum of r and s is ≧3;
t is an average number from 0 to 10;
v is an average number from 1 to 10;
the sum of v and t is ≧3;
Y is a group of the formula (a) or (b) 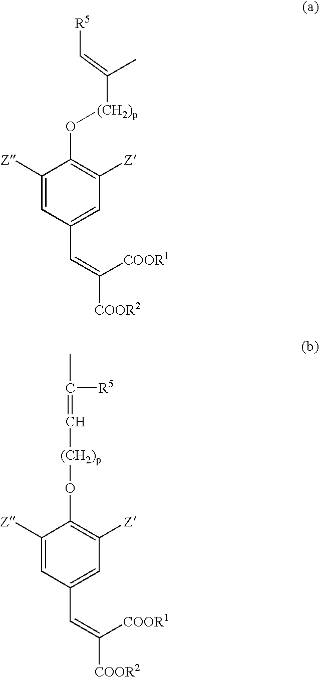
wherein
R¹ and R² are C₁₋₆alkyl;
R⁵ is hydrogen or C₁₋₆alkyl;
Z′ and Z″ are, independently, hydrogen, hydroxy, C₁₋₆alkyl or C₁₋₆alkoxy; and
p is an integer from 1 to 6;
or a group of the formula (c) 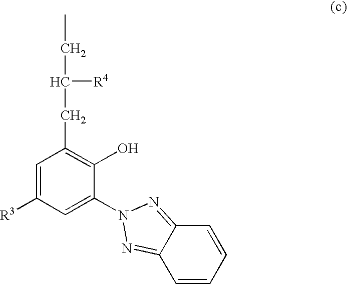
wherein R³, R⁴ is hydrogen or C₁₋₆-alkyl.

14. A method according to claim 12 or 13 wherein the sunscreen is of the formula 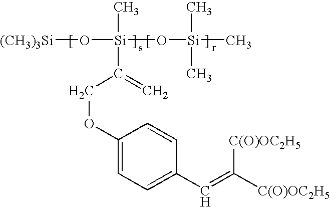
wherein s is about 4 and r is about 60, and which contains about 20% of the alkylene isomer of formula (b).

15. A method according to claim 12 wherein the sunscreen absorbs in a range selected from the group consisting of UV-A, UV-B, broadband and mixtures thereof.

16. A method according to claim 12 wherein the amount of the sunscreen in the composition is from about 0.01 to about 10 wt.-% of the total weight of the composition.

17. A method according to claim 13 wherein the amount of the sunscreen in the composition is from about 0.01 to about 10 wt.-% of the total weight of the composition.

18. A method according to claim 14 wherein the amount of the sunscreen in the composition is from about 0.01 to about 10 wt.-% of the total weight of the composition.

19. A method according to claim 15 wherein the amount of the sunscreen in the composition is from about 0.01 to about 10 wt.-% of the total weight of the composition.

20. A method according to claim 12 wherein the composition is a hairspray.
